(19) United States
(12) Patent Application Publication
(10) Pub. No.: US 2019/0317079 A1
(43) Pub. Date: Oct. 17, 2019

(54) SYSTEM AND METHOD FOR VOLATILE ORGANIC COMPOUND DETECTION

(71) Applicant: SIGHTLINE INNOVATION INC., Toronto (CA)

(72) Inventors: Wallace TRENHOLM, Toronto (CA); Mark ALEXIUK, Winnipeg (CA); Hieu DANG, Winnipeg (CA); Siavash MALEKTAJI, Winnipeg (CA)

(21) Appl. No.: 16/161,458

(22) Filed: Oct. 16, 2018

Related U.S. Application Data
(60) Provisional application No. 62/572,600, filed on Oct. 16, 2017.

(51) Int. Cl. G01N 33/52; G01N 21/31; G16C 20/70; G06N 20/00; G06T 7/00

(52) U.S. Cl. CPC G01N 33/52; G01N 21/31; G06T 2207/20224; G06N 20/00; G06T 7/0002; G16C 20/70

(57) ABSTRACT

A system and method for identifying an analyte based on the presence of at least one volatile organic compound (“VOC”) in the analyte. The method includes: receiving image data from a sensor array, the sensor array having been exposed to the analyte, the sensor array including at least one sensor configured to respond to the presence of the at least one VOC in the analyte; processing the image data to derive one or more input image features; and using a trained machine learning classification technique, detecting the at least one VOC and classifying the analyte based on the one or more input image features, the machine learning classification technique trained using one or more reference images of known analytes.

### TECHNICAL FIELD

The present disclosure relates generally to image processing. More particularly, the present disclosure relates to a system and method for detecting volatile organic compounds in an analyte.

### BACKGROUND

Volatile organic compounds (“VOCs”) are organic compounds that can change, often easily, into a gaseous state at ambient temperatures and pressure conditions. VOC molecules generally include hydrogen, oxygen, fluorine, chlorine, bromine, sulfur and nitrogen in addition to carbon, and can be found in nature, such as in oil and gas fields, and as naturally occurring rubber and resin in plants. VOCs can also be released from man-made products such as paints, inks, solvents, glues, perfumes, cleaning products, landfills, treatment plants, fuel combustion and from effluents from factories and refineries. Many VOCs are hazardous air pollutants and can be lethal in high concentrations. VOCs pose serious health concerns as they can exhibit toxic, carcinogenic, mutagenic and neurotoxic properties. Short term VOC exposure may cause fatigue, headache, dizziness, itchy eyes, skin irritation, shortness of breath, and other symptoms, while long term exposure may result in damage to the nervous system and organs such as the liver and kidneys. Public health agencies have created regulations and solvent emissions directives that limit the emissions of VOCs into indoor and outdoor air.

The presence of VOCs in human breath and urine has great potential for medical, toxicological, forensic and homeland security applications. Studies have indicated that VOCs in humans correlate well with some diseases, metabolic disorders and frequent exposure to toxic environmental contaminants including chemical, biological and radiological agents.

VOCs can also be found in warfare agents such as chemical weapons and explosives. These can include nerve agents, vesicants and poisons. Explosives and chemical weapons are highly reactive and can include compounds such as phosgene, cyanides, organic peroxides, nitrated aromatic and aliphatic compounds, fuel oxidizer mixtures, and the like. These compounds and products created from their degradation over time may release VOCs. This property can be exploited to detect chemical weapons and explosives if the constituting VOCs can be detected and identified.

Existing technologies for detecting VOCs, such as electronic noses, have limitations; for example: sensitivity to oxygen containing compounds, ammonia, carbon-dioxide, high operating temperatures, sensitive to humidity, bulky, complex setup, requirement of controlled environment, limited shelf life, lack of reusability due to permanent denaturing of certain elements upon single exposure to certain VOCs, complex circuitry, and baseline drift, among others. Such technologies can have high operational expenses, maintenance costs, and training costs, as well as complex operational procedures making them unsuitable for large scale deployment.

Accordingly, a system and method for detecting VOCs is desired that alleviates limitations of current methods and systems, such as through the application of machine learning techniques to image data.

### SUMMARY

In an aspect, there is provided a method of identifying an analyte based on the presence of at least one volatile organic compound (“VOC”) in the analyte, comprising: receiving image data from a sensor array after the sensor array has been exposed to the analyte, the sensor array comprising at least one sensor configured to respond to the presence of the at least one VOC in the analyte; processing the image data to derive one or more input image features; and using a trained machine learning classification technique, detecting the at least one VOC and classifying the analyte based on the one or more input image features, the machine learning classification technique trained using one or more reference images of known analytes.

In a particular case, the sensor array comprises a colorimetric sensor array of a plurality of colorimetric sensors, each colorimetric sensor changing in color or in color intensity when exposed to the VOC present in the analyte.

In another case, receiving the image data comprises repeatedly receiving image data of the sensor array at a series of time intervals.

In yet another case, processing the image data to derive the one or more input image features comprises comparing image data in each of the images in the image series to the one of the reference images to generate comparison image data comprising color differences, the input image features comprising the comparison image data.

In yet another case, for each image in the image series, processing the image data comprises: performing edge detection on the image data; performing a Hough circle transform on the image data; detecting an image rotation angle; and where there is a previous image in the image series, aligning the image with the previous image.

In yet another case, processing the image data comprises applying adaptive thresholding to produce a binarized image of the image data.

In yet another case, processing the image data further comprises performing a Hough Circle Transform to detect a blob circle on the binarized image and to segment color blobs on the binarized image.

In yet another case, processing the image data further comprises predicting missing a missing color blob using geometric interpolation.

In yet another case, the one or more input image features comprise color features comprising at least one of global mean, global mode, inner mean, and inner mode.

In yet another case, the one or more input image features comprise textural features comprising at least one of co-occurrence matrix, angular second moment, contrast, correlation, entropy, Hellinger Distance, and Hausdorff Distance.

In yet another case, the machine learning classification technique comprises one of principal component analysis, support vector machines, stacked auto encoders, multi-layer perceptrons, recurrent neural networks, or deep learning neural networks.

In another aspect, there is provided a system for identifying an analyte based on the presence of at least one volatile organic compound (“VOC”) in the analyte, the system in communication with an image acquisition device, the system comprising one or more processors in communication with a memory, the one or more processors configured to execute: an image processing module to: receive image data from a sensor array on the image acquisition device after the sensor array has been exposed to the analyte, the sensor array comprising at least one sensor configured to respond to the presence of the at least one VOC in the analyte; and process the image data to derive one or more input image features; and a classification module to, using a trained machine learning classification technique, classify the analyte based on the one or more input image features, the machine learning classification technique trained using one or more reference images of known analytes.

In a particular case, the sensor array comprises a colorimetric sensor array of a plurality of colorimetric sensors, each colorimetric sensor changing in color or in color intensity when exposed to the VOC present in the analyte

In another case, receiving the image data comprises repeatedly receiving image data of the sensor array at a series of time intervals.

In yet another case, processing the image data to derive the one or more input image features comprises comparing image data in each of the images in the image series to the one of the reference images to generate comparison image data comprising color differences, the input image features comprising the comparison image data.

In yet another case, processing the image data comprises applying adaptive thresholding to produce a binarized image of the image data.

In yet another case, processing the image data further comprises performing a Hough Circle Transform to detect a blob circle on the binarized image and to segment color blobs on the binarized image.

In yet another case, the one or more input image features comprise color features comprising at least one of global mean, global mode, inner mean, and inner mode.

In yet another case, the one or more input image features comprise textural features comprising at least one of co-occurrence matrix, angular second moment, contrast, correlation, entropy, Hellinger Distance, and Hausdorff Distance.

In yet another case, the machine learning classification technique comprises one of principal component analysis, support vector machines, stacked auto encoders, multi-layer perceptrons, recurrent neural networks, or deep learning neural networks.

These and other aspects are contemplated and described herein. It will be appreciated that the foregoing summary sets out representative aspects of systems and methods to assist skilled readers in understanding the following detailed description.

### BRIEF DESCRIPTION OF THE DRAWINGS

The features of the invention will become more apparent in the following detailed description in which reference is made to the appended drawings wherein:

FIG. 1 is a block diagram of an analyte identification system, in accordance with an embodiment;

FIG. 2A is a flowchart of a method for identifying an analyte based on the presence of one or more volatile organic compounds, in accordance with an embodiment;

FIG. 2B is a schematic diagram of a method of analyte detection using a reference sensor array, in accordance with an embodiment;

FIG. 3 is a method for processing a colorimetric sensor array (“CSA”) image, in accordance with an embodiment;

FIG. 4A is a method for processing a CSA image, in accordance with an embodiment;

FIG. 4B is a method for processing a CSA image, in accordance with an embodiment;

FIG. 5A is a method for processing a CSA image, in accordance with an embodiment;

FIG. 5B is a method for processing a CSA image, in accordance with an embodiment;

FIG. 5C is an exemplary image of indexed individual colorimetric sensors on a CSA, in accordance with an embodiment;

FIG. 6 is an exemplary distribution for determining color kernel density mode for a colorimetric sensor, in accordance with an embodiment;

FIG. 7 is an exemplary image showing a variation in response across a colorimetric sensor and resulting multimodal distribution, in accordance with an embodiment; and

FIG. 8 is a general overview of a method for processing a CSA image, in accordance with an embodiment.

### DETAILED DESCRIPTION

Embodiments will now be described with reference to the figures. For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

Any module, unit, component, server, computer, terminal, engine or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

The following relates generally to image processing, and more particularly to the detection of volatile organic compounds (“VOCs”) in an analyte through application of machine learning techniques to image data.

VOCs can be detected based on different principles and interactions between organic compounds and sensor elements. For example, some devices can detect total ppm concentrations without any specificity whereas others can predict molecular structure of a VOC with reasonable accuracy. Some currently commercially available VOC sensors include electrochemical sensors, metal oxide semiconductor sensors, infrared sensors, thermal sensors, flame ionization sensors, and photo ionization sensors. Further VOC sensor types include gas chromatography, mass spectrometry, chemiresistors, surface acoustic wave, optical, quartz, field effect transistors, hybrid nanostructures and micro electro mechanical system sensors.

A further type of sensor that can be used for detecting the presence of VOCs is based on color changing indicators. Such sensors are referred to as “colorimetric sensors” and may use an array of thin films of chemically responsive dyes in incrementally varied compositions to produce a measurable color change, for example when exposed to an analyte comprising VOCs and compounds such as acids, alcohols, aldehydes, amines, arenes, ketones, thiols, and the like. The chemoresponsive sensor elements may comprise metalloporphyrins, base indicators, acid indicators, redox dyes, solvatochromic dyes, nucleophilic indicators, or the like. The colorimetric sensor array (“CSA”) response, depending on the type of substrate used, can be either change in intensity of the color or change of the color itself.

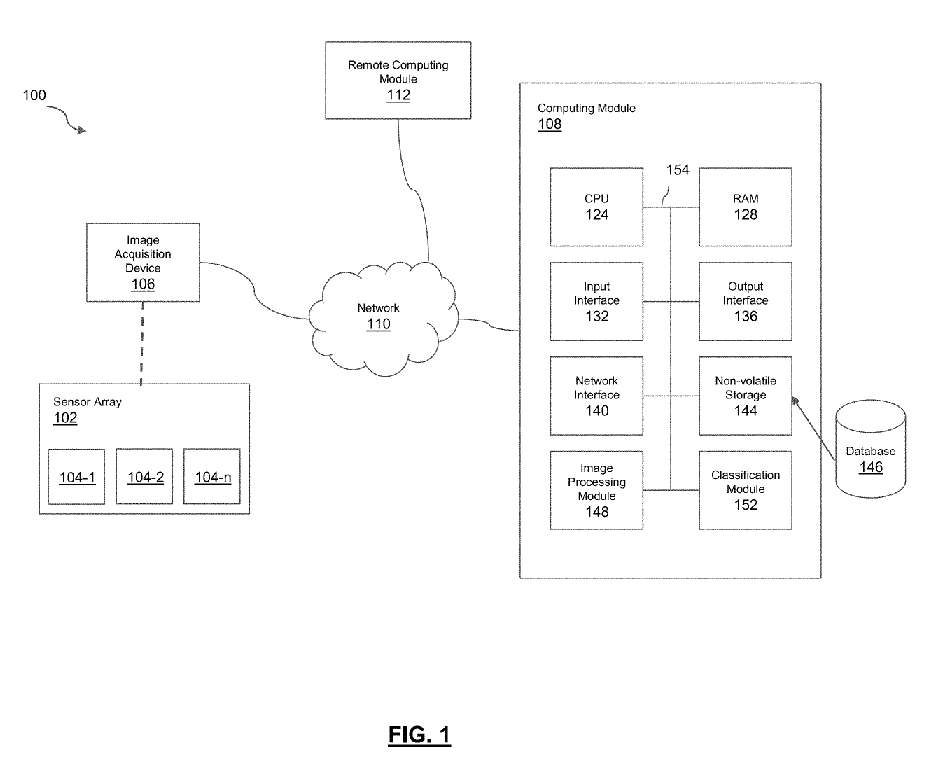

Referring now to FIG. 1, shown therein is an analyte identification system 100, in accordance with an embodiment. The system 100 comprises a sensor array 102 and a machine vision system for identifying an analyte provided to the system 100. The machine vision system comprises an image acquisition device 106 for acquiring images of the sensor array 102, and a computing module 108 for receiving the images from the image acquisition device 106 and processing the images and related data according to image processing and machine learning techniques. In some cases, computing module 108 may process and analyze the image data according to a classification technique. The computing module 108 may be locally communicatively linked or remotely communicatively linked, for example via a network 110, to the image acquisition device 106. The computing module 108 may be used for processing and analysis of image data acquired by the image acquisition device 106. The computing module 108 may host a user-accessible platform for invoking services, such as reporting and analysis services, and for providing computational resources to effect machine learning techniques on the imaging data. In some embodiments, the system 100 may exclude the image acquisition device 106, sensor array 102, or both. In such embodiments, outputs from a sensor array and/or image acquisition device may be provided to computing module 108 from a database, external storage, other device, or the like, to perform data processing and analysis. Generally, the computing module 108 is configured to detect the presence of one or more VOCs in the analyte and may, in some cases, use this determination to identify the analyte based on the detected VOCs.

In a particular case, the machine vision system measures the response of the sensor array 102 over time by imaging the sensor array 102 exposed to the analyte at intervals and compares the image data to a reference sensor array. In some cases, the sensor array 102 is imaged prior to and after exposure to the analyte. In a particular embodiment, the sensor array 102 is imaged at one hour intervals for a period of 24 hours. The computing module 108 may correlate a difference in intensity of individual sensors in the sensor array 102 to constituents, which may then be further correlated to the parent material responsible for producing the detected VOCs.

The sensor array 102 may comprise a plurality of sensors each configured to respond to the presence of a particular VOC; for example, a first sensor 104-1 configured to respond to the presence of a first VOC in the analyte, and a second sensor 104-2 configured to respond to the presence of a second VOC in the analyte. A single sensor may be configured to respond to multiple VOCs, and multiple sensors may be configured to respond to the same VOC. Sensors in the sensor array 102 may produce a binary or continuous output. Collectively, sensors 104-1, 104-2, and 104-n are referred to as sensors 104, and generically as sensor 104 or individual sensor 104.

In an embodiment, the sensor array 102 is a colorimetric sensor array (“CSA”). The CSA 102 is based on color change as an indicator. The CSA 102 may comprise a plurality of individual sensors 104 arranged in a geometric configuration. In a particular case, the sensors 104 are arranged along a horizontal plane to allow uniform exposure to the analyte. Geometric configurations may include, without limitation, a rectangular or a square grid where individual sensors 104 are uniformly arranged along x- and y-axes on a thin slide, or in a circular petri dish configuration where individual sensors are radially distributed in one or more concentric circles. In a further embodiment, a plurality of colorimetric sensors may be distributed in a three dimensional space to allow exposure of sensors 104 to the analyte while allowing for gravity and buoyancy to naturally separate the constituents of analyte along the vertical axis. The CSA response being measured may depend on the type of substrate used and can include a change in intensity of a color, a change of the color itself, and the like.

The image acquisition device 106 can be any suitable device for acquiring an image of the sensor array 102 and its type is not particularly limited. For example, the image acquisition device 106 may be a point scanner, a flatbed or pass-through scanner, an area scan camera, a line scan camera, or the like. The image acquisition device 106 may be configured to operate in the visible color spectrum or in multi-spectral space. The image acquisition device 106 can be stationary (e.g. mounted to stand at a fixed distance) or portable (e.g. handheld mobile device). In some cases, the image acquisition device 106 may comprise a designated imaging device, such as a single-lens reflex camera used by professional photographers, or may be part of a multipurpose device such as a handheld mobile device or computer tablet. The image acquisition device may be capable of capturing time-lapse images at pre-programmed intervals, or may allow for manual imaging at an operator's request.

The image acquisition device 106 may be capable of embedding exchangeable image file format (EXIF) information in the acquired digital image of the CSA. EXIF data may include: make and model of camera, date, time, resolution, exposure, aperture, F-number, lens-type, focal length, color space, image compression, metering mode, camera flash, and the like. Such information may be used for the purposes of image calibration.

In a particular example, the CSA 102 is a 1.2 cm×2.1 cm patterned CSA spotted with 80 “features,” with each spot being the equivalent of 1.5-2.0 mm in diameter. Of the 80 features, 73 are responsive with the remainder comprising indicators for alignment and control. Each spot includes a fluorescent dye responsive to vapours and possibly radiation. The CSA comprises an array of colored chemical indicators of diverse reactivities embedded in a nanoporous sol-gel matrix. Each indicator in the array sensitively changes color, creating a high dimensional and specific fingerprint allowing identification of the species or mixture present. Because the reactive indicators are highly diverse chemically, a wide range of chemical species can be uniquely detected.

In such an embodiment, the detection event can be observed as a plurality of printed dots changing color. These detection events can be recorded with image acquisition device 106. While kinetic information may be obtained by repeatedly scanning the CSA with the image acquisition device 106, in some cases images may simply be taken before and after exposure of the CSA to the analyte. From the two images of the CSA—the before exposure image and after exposure image—a color difference map can be generated, such as by superimposing and subtracting the two images. The color difference map can be quantified as a high dimensional vector of RGB values. The high dimensional vectors can be used to discriminate between analytes while retaining their chemical resemblances.

In a particular case, the machine vision system captures an image of each sensor color. The image may be quantized in an 8-bit image format with a value between 0 and 255 for each of the three color channels (red, green, and blue). This data may be collected over a regular interval of time; for example, 24 readings of the CSA taken at one hour intervals. This can quickly add up to a large number of data points that can serve as a characteristic digital signature of the analyte. A CSA with 76 sensors imaged at one hour intervals for 24 hours can produce a digital signature with over 5000 data points (i.e. 76 sensors×3 color channels×24 time intervals) for a given analyte.

In an embodiment, as shown in FIG. 1, the computing module 108 can include a number of physical and logical components, including a central processing unit (“CPU”) 124, random access memory (“RAM”) 128, an input interface 132, an output interface 136, a network interface 140, non-volatile storage 144, and a local bus 154 enabling CPU 124 to communicate with the other components. CPU 124 can include one or more processors. RAM 128 provides relatively responsive volatile storage to CPU 124. The input interface 132 enables an administrator to provide input via, for example, a keyboard and mouse. The output interface 136 outputs information to output devices, for example, a display or speakers. The network interface 140 permits communication with other systems or computing devices. Non-volatile storage 144 stores the operating system and programs, including computer-executable instructions for implementing the system 100 or analyzing data from the image acquisition device 106, as well as any derivative or related data. In some cases, this data can be stored in a database 146. During operation of the computing module 108, the operating system, the programs and the data may be retrieved from the non-volatile storage 144 and placed in RAM 128 to facilitate execution. In an embodiment, the CPU 124 can be configured to execute various modules, for example, an image processing module 148 and a classification module 152.

As described above, use of the sensor array 102 (such as a CSA) can generate a large amount of data. Specialized techniques may be required to perform analysis on data. In some cases, machine learning (“ML”) techniques such as neural networks and statistical pattern recognition may be used to process and analyze the data. In a particular embodiment, ML techniques can be used to develop a reference databank comprising known analytes. The reference databank can be compared with a CSA output from an unknown analyte sample in order to identify the analyte, such as according to a classification process.

In an embodiment, the image processing module 148 performs one or more image processing tasks such as image rotation, alignment, segmentation, white balance adjustment, color correction, blob detection, edge detection, sharpening, smoothing, geometric interpolation, histogram equalization, image normalization, registration, filtering, thresholding, pixel counting, color analysis, denoising, and the like. Image processing tasks can be performed on image data prior to generating color and textural features for the colorimetric sensors.

The image processing module 148 may generate color and textural features from the CSA images, which can be used by the classification module 152 for the purpose of identifying the analyte. “Texture” as used here refers to visual texture rather than physical texture. Textural features can provide information about the spatial arrangement of color or intensities in the image or a selected region of the image. In an embodiment, textural features may include distance and covariance functions such as co-occurrence matrix, angular second moment, contrast, correlation, entropy, Hellinger Distance, the Hausdorff Distance, and the like.

Color features can play an important role in classification. Color features can include global mean, global mode, inner mean, inner mode, and the like. “Global” values of the sensor response can be calculated by computing the color data over all the pixels, while “inner” values can be calculated by computing features using only pixels that are within a certain distance from the centroid. In some cases, extracting color features can require an accurate and robust blob detection method.

In an embodiment, the classification module 152 is configured to implement one or more machine learning (ML) techniques such as principal component analysis, support vector machines, stacked auto encoders, multi-layer perceptrons, recurrent neural networks, deep learning neural networks, and the like, in order to train and develop a classifier for detecting VOCs and identifying the analyte. The ML techniques can be applied to first train one or more classifiers using supervised, unsupervised, or semi-supervised learning techniques. Cross-validation techniques may be applied to reduce prediction error and derive a more accurate estimate of classifier performance. The classifier can be trained to identify the unknown analyte based on image features, such as color and textural features, computed from the CSA images acquired prior to analyte exposure and after analyte exposure.

In a particular embodiment, a recurrent neural network may be used to develop a classifier from time-lapse images of the CSA 102. The time-lapse images provide information regarding changes in color and textural features over time upon exposure to the analyte. The recurrent neural network may comprise architectures including but not limited to a fully recurrent network, a recursive neural network, a Hopfield network, an Elman-Jordan network, a Long short-term memory, a Gated recurrent unit, or the like.

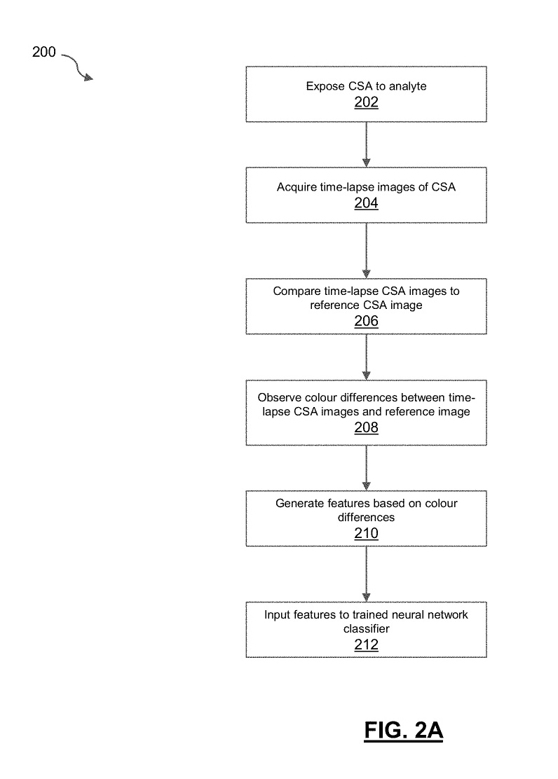

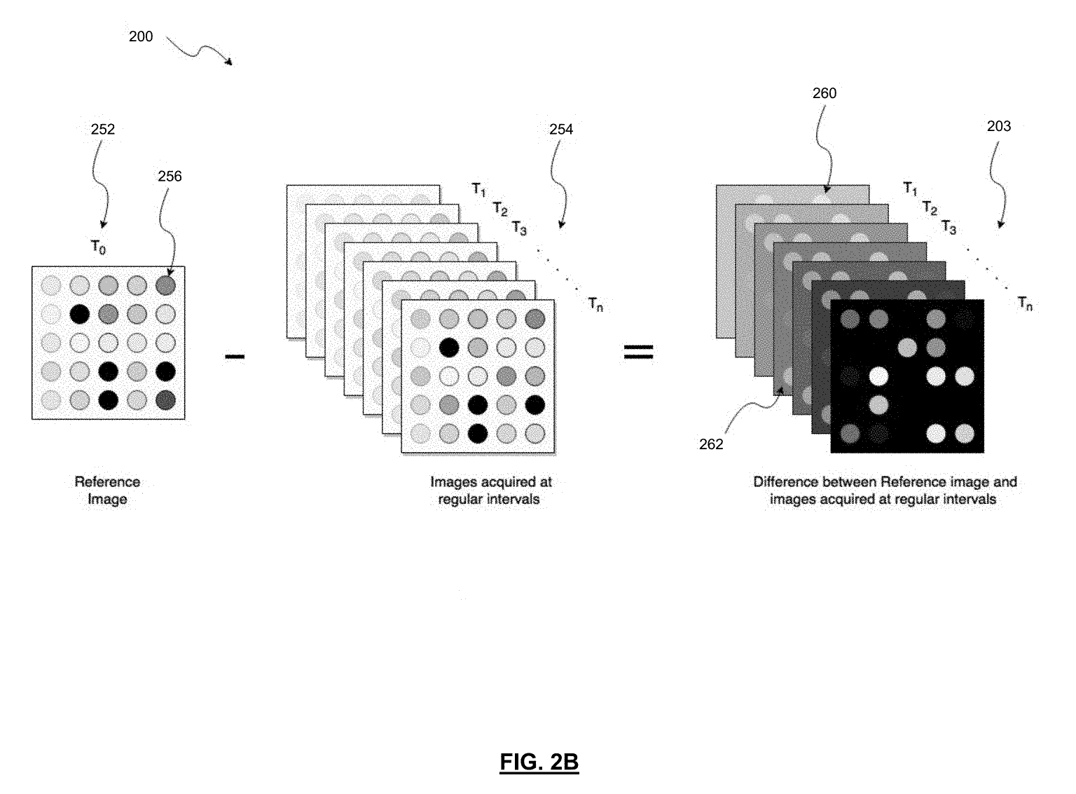

Referring now to FIG. 2A, shown therein is a method 200 for identifying an analyte using a reference image, in accordance with an embodiment. Method 200 can be used to generate input features for a trained classifier, such as a neural network. In the particular embodiment shown in FIG. 2, the sensor array 102 is a CSA. At block 202, the CSA is exposed to the unknown analyte. At block 204, images are taken of the exposed CSA at a series of time intervals (e.g. T₁, T₂, . . . , Tₙ). In some cases, the CSA may be imaged prior to its exposure to the analyte. At block 206, each image in the CSA image series is compared to a CSA reference image in order to generate a comparison image comprising color differences between the reference image and the acquired image. At block 208, the color differences are computed. At block 210, the image processing module 148 generates features based on the color differences that can be used as input for a classification process. At block 212, the input features are provided to a trained neural network at an input layer for classification of the analyte.

FIG. 2B shows a schematic representation of the method 200 of FIG. 2A. A reference CSA image 252 is used as a comparator. Time-lapse images 254 of the CSA after exposure to the analyte are collected. Changes to individual colorimetric sensors 256 of the CSA can be measured over time as a response to analyte exposure. Each time-lapse image 254 is compared with the reference image 252 to generate a comparison image 258 comprising the color changes to individual colorimetric sensors 256 over time. The color differences computed in the comparison image 258 can serve as an input feature to a classifier, such as a trained neural network. Depending on the analyte and the concentration of various VOCs in the analyte, a variable number of individual colorimetric sensors 256 may react with the VOCs present in the analyte. For individual colorimetric sensors 256 that react to the presence of a VOC, a change in color may be dependent on reactivity of the individual colorimetric sensor 256 to certain types of VOCs (e.g. acids, alcohols, aldehydes, amines, arenes, ketones, thiols, and the like), and on the duration of exposure of the colorimetric sensor to the VOC in the analyte. Individual sensors 256 of the CSA may react differently to the presence of VOCs in the analyte. For example, certain colorimetric sensors may not react to VOCs. Such nonreactive colorimetric sensors are not visible in the comparison image 258. In other instances, colorimetric sensors may react right away and the color difference is visible in the T₀ image, such as with sensor 260; while other colorimetric sensors may react slowly, producing a color difference that is visible only upon significant passage of time, such as with sensor 262.

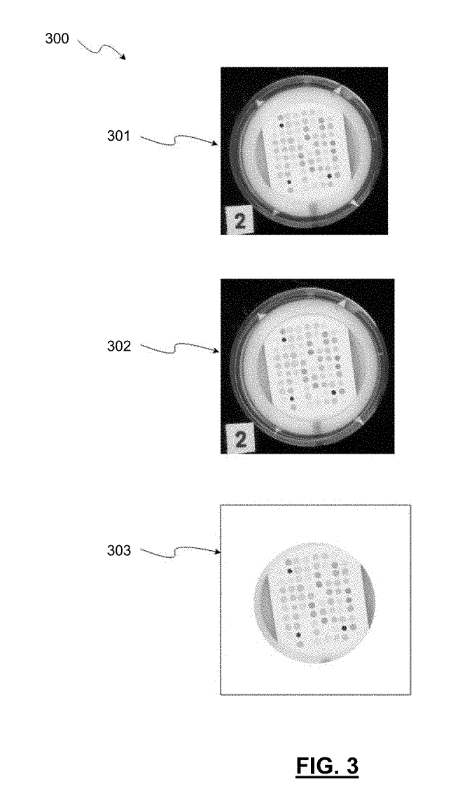

Referring now to FIGS. 3 to 5, shown therein are example methods and implementations of image processing techniques for the systems and methods described herein. Various steps of the methods and implementations described below may be carried by computing module 108, and particularly by image processing module 148. Images and image data may be provided to the computing module 108 by image acquisition device 106, or from a historical database of images (e.g. database 146).

Referring now to FIG. 3 shown therein is an image processing sequence 300 for segmenting an image 301, in accordance with an embodiment. The image 301 comprises an image of a CSA that has been exposed to an analyte. Depending on the configuration and arrangement of the image acquisition device 106, the field of view may capture more information than required. For example, image 301 shows an image taken of the CSA that includes more information (i.e. background) than necessary. In a particular embodiment, the CSA is housed in a circular container that can be identified using a Hough circle transform, as shown in image 302. The Hough circle detection technique works by detecting points in image 301, some of which may fall on perimeter of circles. The speed of the process can be increased by eliminating unwanted points in the image and running the search for circles on points that comprise an edge of an object in the image. Therefore, prior to running the Hough circle transform, an edge may be detected using an edge detection technique, such as a Canny edge detector. In some cases, depending on circle detection parameters, the Hough circle transform may detect several circles in the image, as shown by the light circular lines in image 302. In the present case, the smallest detected circle in image 302 is of interest and can be selected to produce a segmented image 303 of the CSA that can be used for further processing. Where the CSA includes individual colorimetric sensors that are circular in shape, circle detection parameters can be set such that the individual colorimetric sensors are ignored at this stage.

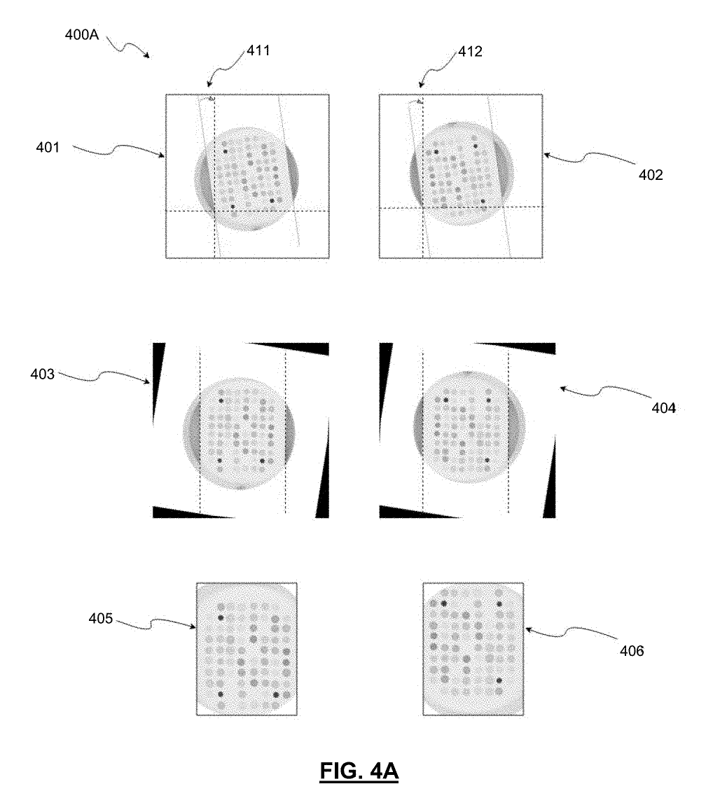

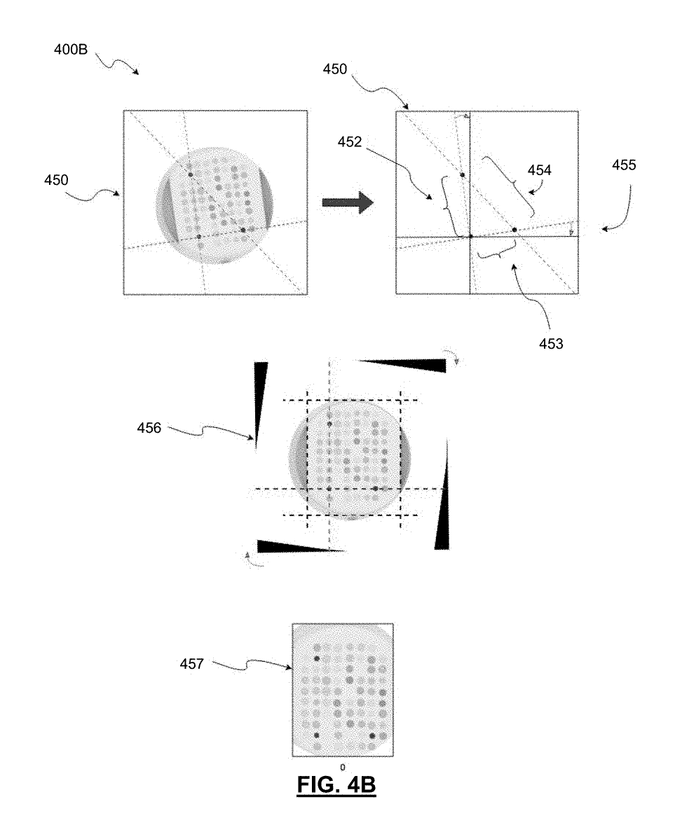

Referring now to FIG. 4A, shown therein is an image processing sequence 400A for detecting an image rotation angle in order to align a CSA contained in the image (e.g. in a portrait mode). An edge detection technique is used to detect linear edges in the previously segmented image 303 (as shown in images 401, 402). Linear edges may be detected using techniques such as Sobel, Canny, and Hough edge detector. Having detected linear edges in the image, the rotation angle (411, 412) can be determined. In a particular case, this is done in a manner such that the angular transformation of the image would result in the linear edges being vertical (as shown in images 403, 404). It may be noted however that depending on the initial orientation of the image 303, using just an edge detector may result in two possible portrait orientations of the CSA, one rotated at 180 degrees with respect to the other (as shown in images 405, 406). To address this issue, fiducial markers can be used that may be present on the CSA and are not invariant to rotation.

Referring now to FIG. 4B, shown therein is an image processing sequence 400B for aligning a segmented image using fiducial markers, in accordance with an embodiment. For example, the image processing sequence 400B can be used to align the previously segmented CSA image 303. The CSA shown in image 450 includes three fiducial markers (the darkest dots on the CSA). The fiducial markers do not react or change color when exposed to analytes (or any VOCs contained therein). As shown in 451, adaptive thresholding can be applied to image 450 to segment the three fiducial markers and determine the length of the sides 452, 453, 454 of a triangle connecting the fiducial markers. The image 450 is rotated at the intersection of the shortest side 453 and intermediate side 452, such that the shortest side 453 is horizontal and intermediate side 452 is vertical. This ensures that the resulting rotated images 456 are aligned with each other. The rotated image 456 can be cropped along the vertical edges to remove background to produce a cropped image 457 that can be used further image processing steps.

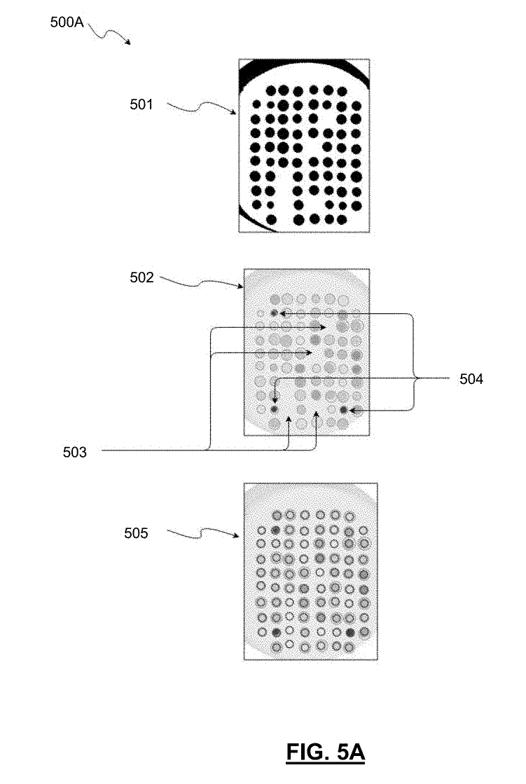

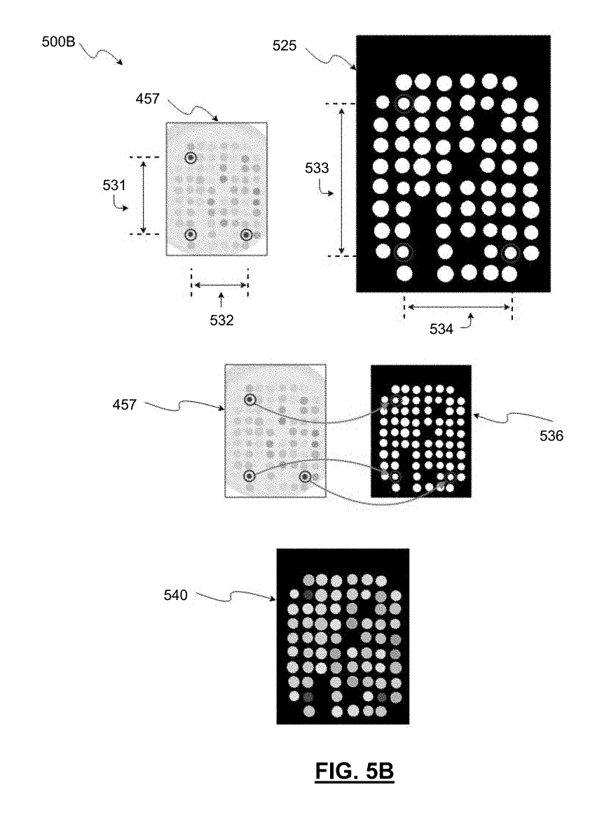

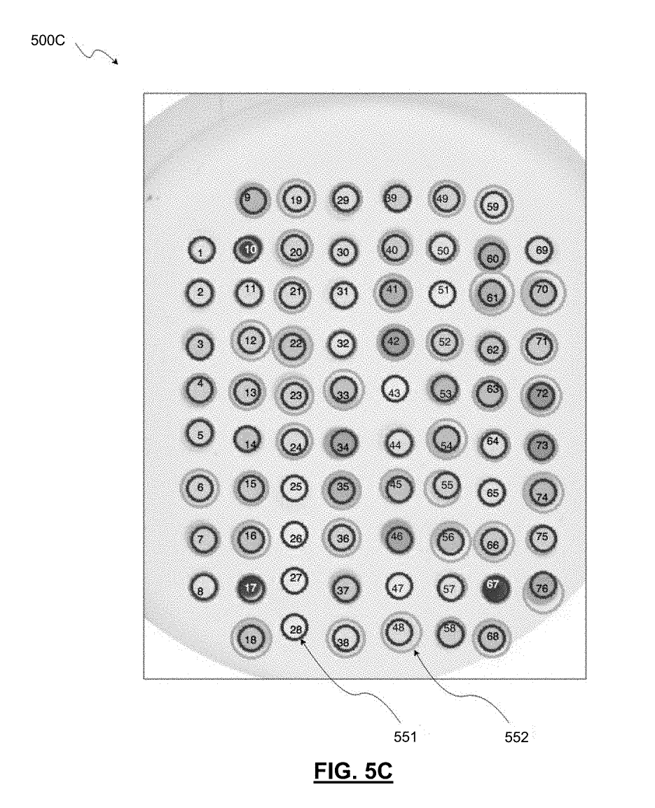

Referring now to FIG. 5A, shown therein is an image processing sequence 500A for segmenting individual colorimetric sensors on a colorimetric sensor array, in accordance with an embodiment. The image processing sequence 500A can be applied to cropped image 457 of FIG. 4B, and includes applying adaptive thresholding to produce a binarized image 501, blob detection 502, and geometric interpolation 505. Adaptive thresholding is a form of local thresholding that takes into account spatial variations in illumination. Instead of using a single global value to remove image background, the background and foreground are segmented based on local color variations. A Hough circle transform is applied to detect a blob circle on the binarized image 501. On some CSAs, in addition to dark fiducial markers 504, there may be additional sensor-less areas 503 that do not contain a colorimetric sensor. These blank spots 503 can be used to perform alignment, color calibration, adjust white balance, or the like, to normalize all the images and offset any unintended variation prior to extracting features and performing data analysis. In the present case, the individual colorimetric sensors are uniformly distributed. Blank spots 503 can thus be assigned a blob by performing a simple geometric interpolation and predicting a blob location. As shown in image 505, the individual colorimetric sensors can be identified via a first identifier, such as a green circle (appearing in the image 505 as a light circle), and the predicted blobs identified using a second identifier, such as a red circle (appearing in the image 505 as a dark circle).

Referring now to FIG. 5B, shown therein is an image processing sequence 500B for segmenting individual colorimetric sensors on the CSA, in accordance with an embodiment. A binary mask 525 is applied to cropped image 457 (from FIG. 4B) to produce a segmented image 540. Prior to applying the binary mask, the binary mask image can be scaled to match the CSA image 457. This can be achieved by determining a horizontal distance 532 and a vertical distance 531 between the fiducial markers on the CSA, and comparing them to a horizontal distance 534 and a vertical distance 533 between the fiducial markers on the binary mask. The binary mask is scaled horizontally by a value of 532/534, and vertically by a value of 531/533 to produce a scaled binary mask 536 such that it aligns with the CSA image 457. The scaled binary mask 536 can be applied to CSA image 457 in order to generate image 540. Image 540 can be segmented and detected blobs can be indexed.

Referring now to FIG. 5C, shown therein is an exemplary image 500C of a CSA having indexed individual colorimetric sensors, in accordance with an embodiment. The individual colorimetric sensors are identified by a lighter circle 552, while the predicted blobs are identified by a darker circle 551. Fiducial markers are shown at index location 10, 17 and 67; sensorless areas, as determined by geometric interpolation or a binary mask, occupy index locations 25, 26, 27, 28, 43, 47, and 51.

Based on the detected and interpolated color blobs for the colorimetric sensors from the image processing steps, all the pixels in the blob circle can be acquired. For each color blob, color and textural features can be computed; for example, a mean and mode for each of red, green and blue color pixels. In an embodiment using a CSA having 76 colorimetric sensors, an analyte that is imaged every hour for a 24 hour period can produce a feature vector comprising 24 labeled feature vectors of size 76×3=228 values of color mean differences and 228 values of color mode differences. Thus, in such a case the size of the feature vector for one feature (e.g. color mean) over 24 time lapse images is 24×228=5472. For two features (e.g. color mean and color mode), the size of the feature vector over 24 time lapse images would be 24*228*2=10,944, and so forth. The size of the input feature vector increases dramatically with an increase in the number of time lapse images and number of features (whether color, textural, or other). Techniques such as principal component analysis can be performed to reduce the dimensionality of data, which may in turn help reduce variance of a predictor or classifier, such as a support vector machine.

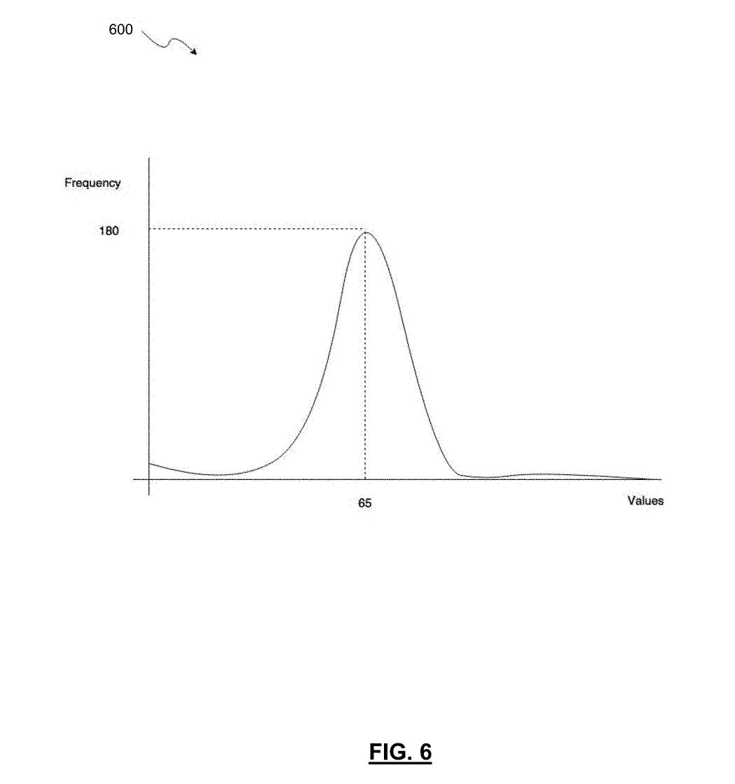

Referring now to FIG. 6, shown therein is an exemplary distribution 600 for determining color kernel density mode for a colorimetric sensor, in accordance with an embodiment. Finding color kernel density mode involves finding the dominated color value in an area or an array. Colorimetric sensors are ideally expected to generate a uniform color change across a colorimetric sensor upon reacting with an analyte, producing a narrow histogram. In distribution 600, a color value of 65 dominates other color values by appearing 180 times in a blob. This may not always be the case as color variations may arise across a colorimetric sensor upon reacting with an analyte due to manufacturing or other causes.

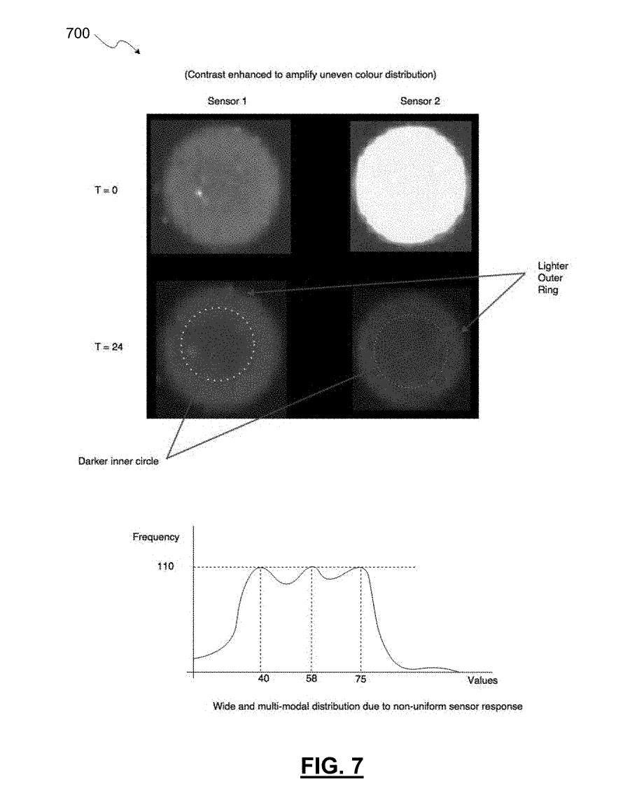

Referring now to FIG. 7, shown therein are exemplary images showing variation in color response for two colorimetric sensors resulting in a multimodal distribution. Colorimetric sensors may have differential responses in the centre of the sensor as compared to the edges, likely due to unevenness of thickness deposition of the CSA. As shown, an image taken 24 hours after exposing the CSA to an analyte shows a relatively darker centre as compared to the outer ring. Plotting a histogram of red, green, or blue color for a given colorimetric sensor may produce a multimodal output as shown. Such a distribution may produce inconsistent input features, which can compromise the effectiveness of the classification process and thus potentially limit accurate identification of the analyte. This may be overcome by using color information from only around the middle of the colorimetric sensor. For example, the color mean and color mode for a colorimetric sensor may be computed using all the pixels that are within a given distance, r/2, from the centre of the colorimetric sensor, where r is the radius of the blob enclosing the colorimetric sensor.

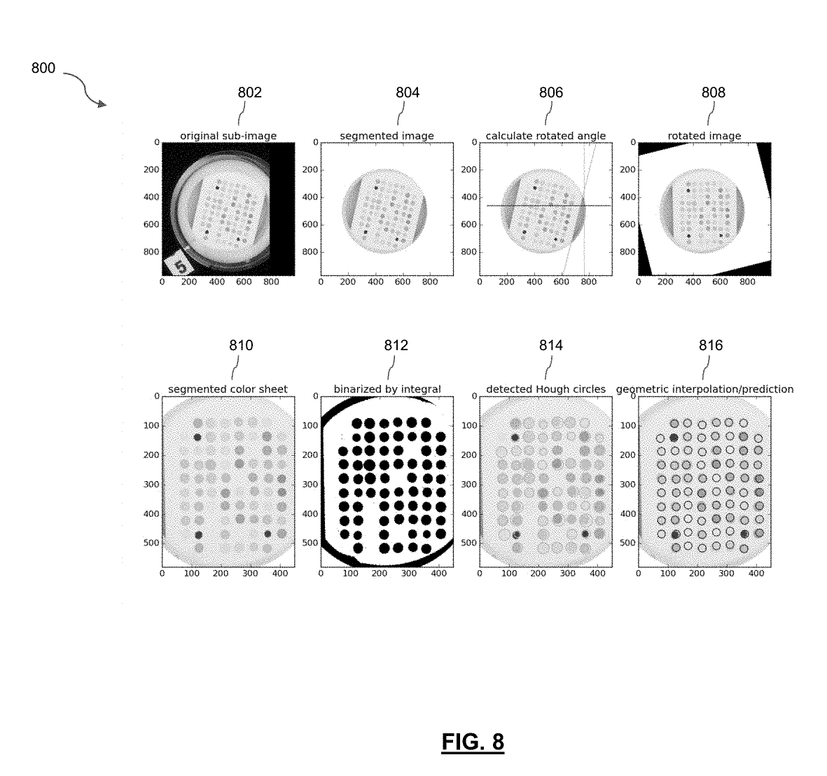

Referring now to FIG. 8, shown therein is an example of an overview of a method 800 for processing a CSA image, in accordance with an embodiment. In the present case, the system has received an image of a plurality of CSAs that have been exposed to an analyte. At 802, the image is segmented to produce a sub-image containing only one CSA. At 804, a Hough Circle Transform is performed on the sub-image to detect the plate circles (preliminary feature extraction). The image is then segmented based on the detected big circle. At 806, a rotation angle of the image is detected and the image is rotated based on the detected rotation angle to produce a rotated image shown at 808. At 810, the color sheet is cropped from the segmented and rotated image based on the detected circle center. At 812, the color sheet is binarized based on the adaptive thresholding based on the image integral algorithm. At 814, an HCT is used again to detect the blob circle on the binarized image and to display the segmented color blobs on the segmented color sheet. At 816, a geometric interpolation algorithm is written to predict missing color blobs. In the present case, the predicted color blobs are displayed in red circles.

Outputs from the image processing techniques can be provided to and used by the computing module 150 in further processing and analysis. For example, machine learning techniques may be applied in order to perform VOC detection and analyte identification.

In an embodiment, the detection of VOCs in an analyte or the identification of the analyte itself and other processing of imaging data for evaluation purposes can be based on computational modules. Computational modules can be implemented using any computational paradigm capable of performing data analysis based on various methods such as regression, classification and others. In some variations, the computational modules can be learning based. One learning based computational paradigm capable of performing such methods may be a neural network. Neural networks may include Restricted Boltzmann Machines, Deep Belief Networks, and Deep Boltzmann Machines. Accordingly, a neural network can be used to identify an analyte based on component VOCs. Thus, feature data such as color and textural feature data, as well as relevant data from databases and other services, can be provided to a neural network, which can perform analyte identification based on classification/regression or similar methods.

In embodiments, analysis and classification by the computing module 108 may be implemented by providing input data to a neural network, such as a feed-forward neural network, for generating at least one output. The neural networks may have a plurality of processing nodes, including a multi-variable input layer having a plurality of input nodes, at least one hidden layer of nodes, and an output layer having at least one output node. During operation of a neural network, each of the nodes in the hidden layer applies an activation/transfer function and a weight to any input arriving at that node (from the input layer or from another layer of the hidden layer), and the node may provide an output to other nodes (of a subsequent hidden layer or to the output layer). The neural network may be configured to perform a regression analysis providing a continuous output, or a classification analysis to classify data. The neural networks may be trained using supervised or unsupervised learning techniques, as described below. According to a supervised learning technique, a training dataset is provided at the input layer in conjunction with a set of known output values at the output layer. During a training stage, the neural network may process the training dataset. It is intended that the neural network learn how to provide an output for new input data by generalizing the information it learns in the training stage from the training data. Training may be effected by back propagating the error to determine weights of the nodes of the hidden layers to minimize the error. Once trained, or optionally during training, test (verification) data can be provided to the neural network to provide an output. A neural network may thus cross-correlate inputs provided to the input layer in order to provide at least one output at the output layer. Preferably, the output provided by a neural network in each embodiment will be close to a desired output for a given input, such that the neural network satisfactorily processes the input data.

The term “classification” as used herein should be understood in a larger context than simply to denote supervised learning. By classification process we convey: supervised learning, unsupervised learning, semi-supervised learning, active/groundtruther learning, reinforcement learning and anomaly detection. Classification may be multi-valued and probabilistic in that several class labels may be identified as a decision result; each of these responses may be associated with an accuracy confidence level. Such multi-valued outputs may result from the use of ensembles of same or different types of machine learning algorithms trained on different subsets of training data samples. There are various ways to aggregate the class label outputs from an ensemble of classifiers; majority voting is one method.

In some embodiments, an ensemble of classifiers may be used, such as multiple support vector machines or other classifiers running simultaneously. The classification ensemble may be homogeneous or heterogeneous. A homogeneous ensemble comprises a plurality of classifiers of the same machine learning type (for example, multiple support vector machines). Each classifier in a homogeneous ensemble may have different parameter values and may be trained using a distinct subset of the samples in the training set. A heterogeneous ensemble comprises a plurality of classifiers belonging to a variety of machine learning algorithms; for example, a deep neural network, a K-means clustering, and an SVM. Classifiers in heterogeneous ensembles may be trained on the same training data or on distinct subsets of the training data. If a multiplicity of a machine learning algorithms exists in a heterogeneous ensemble, each instance of the multiplicity may be trained on some samples unique only to that instance.

In some variations, the neural network can operate in at least two modes. In a first mode, a training mode, the neural network can be trained (i.e. learn) based on known images. The training typically involves modifications to the weights and biases of the neural network, based on training algorithms (backpropagation) that improve its detection capabilities. In a second mode, a normal mode, the neural network can be used to detect VOCs in the analyte using an image of an exposed sensor array. In variations, some neural networks can operate in training and normal modes simultaneously, thereby both detecting the presence of VOCs, and training the network based on the detection effort performed at the same time to improve its detection capabilities. In variations, training data and other data used for performing detection services may be obtained from other services such as databases or other storage services. Some computational paradigms used, such as neural networks, involve massively parallel computations. In some implementations, the efficiency of the computational modules implementing such paradigms can be significantly increased by implementing them on computing hardware involving a large number of processors, such as graphical processing units.

In variations, VOC detection using a neural network or clustering mechanism can be an ongoing process. For example, in some implementations, the computing module can be a local computing module and provide results to a remote computing module. The remote computing module can include appropriate learning mechanisms to update a training model based on the newly received signals. For example, the remote computing module can be a neural network based system implemented using various application programming interfaces APIs and can be a distributed system. The APIs included can be workflow APIs, match engine APIs, and signal parser APIs, allowing the remote computing module to both update the network and determine whether a VOC is present in the analyte.

Embodiments of the systems and methods of the present disclosure may implement groundtruthing to ensure classification result accuracy according to an active learning technique. Specifically, results from classification models may be rated with a confidence score, and high uncertainty classification results can be pushed to a groundtruther to verify classification accuracy. Optionally, classification outputs can periodically be provided to groundtruthers to ensure accuracy. In some implementations, a determination by the system indicative of the presence of a VOC may result in generating a request for human groundtruthing of the detection signal or the sensor array or sensor array image.

Although the invention has been described with reference to certain specific embodiments, various modifications thereof will be apparent to those skilled in the art without departing from the spirit and scope of the invention as outlined in the claims appended hereto. The entire disclosures of all references recited above are incorporated herein by reference.

1. A method of identifying an analyte based on the presence of at least one volatile organic compound (“VOC”) in the analyte, comprising:
receiving image data from a sensor array after the sensor array has been exposed to the analyte, the sensor array comprising at least one sensor configured to respond to the presence of the at least one VOC in the analyte;
processing the image data to derive one or more input image features; and
using a trained machine learning classification technique, detecting the at least one VOC and classifying the analyte based on the one or more input image features, the machine learning classification technique trained using one or more reference images of known analytes.

2. The method of claim 1, wherein the sensor array comprises a colorimetric sensor array of a plurality of colorimetric sensors, each colorimetric sensor changing in color or in color intensity when exposed to the VOC present in the analyte.

3. The method of claim 2, wherein receiving the image data comprises repeatedly receiving image data of the sensor array at a series of time intervals.

4. The method of claim 3, wherein processing the image data to derive the one or more input image features comprises comparing image data in each of the images in the image series to the one of the reference images to generate comparison image data comprising color differences, the input image features comprising the comparison image data.

5. The method of claim 4, wherein, for each image in the image series, processing the image data comprises:
performing edge detection on the image data;
performing a Hough circle transform on the image data;
detecting an image rotation angle; and
where there is a previous image in the image series, aligning the image with the previous image.

6. The method of claim 2, wherein processing the image data comprises applying adaptive thresholding to produce a binarized image of the image data.

7. The method of claim 6, wherein processing the image data further comprises performing a Hough Circle Transform to detect a blob circle on the binarized image and to segment color blobs on the binarized image.

8. The method of claim 7, wherein processing the image data further comprises predicting missing a missing color blob using geometric interpolation.

9. The method of claim 2, wherein the one or more input image features comprise color features comprising at least one of global mean, global mode, inner mean, and inner mode.

10. The method of claim 2, wherein the one or more input image feature comprises textural features comprising at least one of co-occurrence matrix, angular second moment, contrast, correlation, entropy, Hellinger Distance, and Hausdorff Distance.

11. The method of claim 2, wherein the machine learning classification technique comprises one of support vector machines, stacked auto encoders, multi-layer perceptrons, recurrent neural networks, or deep learning neural networks.

12. A system for identifying an analyte based on the presence of at least one volatile organic compound (“VOC”) in the analyte, the system in communication with an image acquisition device, the system comprising one or more processors in communication with a memory, the one or more processors configured to execute:
an image processing module to:
receive image data from a sensor array on the image acquisition device after the sensor array has been exposed to the analyte, the sensor array comprising at least one sensor configured to respond to the presence of the at least one VOC in the analyte; and
process the image data to derive one or more input image features; and
a classification module to, using a trained machine learning classification technique, classify the analyte based on the one or more input image features, the machine learning classification technique trained using one or more reference images of known analytes.

13. The system of claim 12, wherein the sensor array comprises a colorimetric sensor array of a plurality of colorimetric sensors, each colorimetric sensor changing in color or in color intensity when exposed to the VOC present in the analyte.

14. The system of claim 13, wherein receiving the image data comprises repeatedly receiving image data of the sensor array at a series of time intervals.

15. The system of claim 14, wherein processing the image data to derive the one or more input image features comprises comparing image data in each of the images in the image series to the one of the reference images to generate comparison image data comprising color differences, the input image features comprising the comparison image data.

16. The system of claim 13, wherein processing the image data comprises applying adaptive thresholding to produce a binarized image of the image data.

17. The system of claim 16, wherein processing the image data further comprises performing a Hough Circle Transform to detect a blob circle on the binarized image and to segment color blobs on the binarized image.

18. The system of claim 13, wherein the one or more input image features comprise color features comprising at least one of global mean, global mode, inner mean, and inner mode.

19. The system of claim 13, wherein the one or more input image features comprise textural features comprising at least one of co-occurrence matrix, angular second moment, contrast, correlation, entropy, Hellinger Distance, and Hausdorff Distance.

20. The system of claim 13, wherein the machine learning classification technique comprises one of support vector machines, stacked auto encoders, multi-layer perceptrons, recurrent neural networks, or deep learning neural networks.
